(19) United States
(12) Patent Application Publication
(10) Pub. No.: US 2018/0361066 A1
(43) Pub. Date: Dec. 20, 2018

(54) HAND-HELD VOLUME DISPLACEMENT DOSAGE DISPENSER

(71) Applicant: Accugentix, LLC, Naples, FL (US)

(72) Inventor: Alainah M. Hacker, Naples, FL (US)

(21) Appl. No.: 16/012,454

(22) Filed: Jun. 19, 2018

Related U.S. Application Data
(60) Provisional application No. 62/522,568, filed on Jun. 20, 2017.

(51) Int. Cl. A61M 5/20; G16H 20/17

(52) U.S. Cl. CPC A61M 5/20; G16H 20/17; A61M 2205/13; A61M 2205/52; A61M 2205/3576; A61M 2005/2073

(57) ABSTRACT

A portable device that accurately determines the liquid dosage that a patient is consuming while also regulating their use of the device is provided and is suitable for use with substances like medical cannabis and opioids. A liquid dispenser device is reusable with replaceable liquid cartridges. These cartridges can contain multiple dosages of a specific liquid, which is more convenient for the patient and reduces cost. The device uses a micro-motor, translational screw, and encoder to determine the exact amount of liquid displaced from the cartridge. That exact amount of liquid is dispensed from a small nozzle into a mouthpiece. The mouthpiece is replaceable and can be tailored to meet the patient's specific needs. For example, the mouthpiece can be adapted for dispensing under the tongue of the user or directly into a drink. The device can also have locking mechanisms and wireless communication capabilities. The device will not allow patients take over the maximum doctor recommended dosage, and regulates the time interval of those dosages based on doctor recommendation. Wireless communication can be used to communicate with other devices to ensure that the patient is not overlapping their dosages. Wireless communication can also be used to sync the device to a smartphone, tablet, or computer app to track patient data, usage, and the patient can also set a timer/alarm.

### CROSS REFERENCE TO RELATED APPLICATIONS

This application claims benefit of pending U.S. Provisional Application No. 62/522,568 filed Jun. 20, 2017, which is incorporated by reference herein.

### BACKGROUND OF THE INVENTION

This invention relates to a device for the precise measuring and administration of liquids. More particularly, this invention relates to a device for the precise administration of medications or therapeutic liquids. Even more particularly, this invention concerns a portable device for the precise and controlled administration of medications or therapeutic liquids.

Devices that precisely measure and administer a liquid medicine are known in the art. For example, devices designed for a single injection of medicine, utilizing the force of motors, a translational screw, and springs to administer the medicine through injection are known, but these mechanisms do not determine the exact dosage administered to the patient. Multi-use devices may use multiple cartridges within a single device, which makes the device too large to be conveniently portable for patients. These devices are also designed for injection of medicine. These devices do not provide for the oral administration of medications like cannabidiol (CBD) oil, cannabis tincture, opioids, and similar therapeutic liquids.

Many medications are potentially dangerous if not regulated correctly and administered accurately. This is particularly true of medicines based on cannabis and opioids. There is a need for an easy-to-use device that precisely measures a dose of medicine but also ensures that the patient does not exceed a prescribed dosage or repeat dosages in too short a period of time. Such a device would minimize the possibility of overdose or addiction for these potentially dangerous medications. Devices are know which have lock out mechanisms that are meant to regulate who uses the device, how much is taken from the device, and when the device is used. Such a device may also have an interface that the patient can use. But known devices of this type use spring mechanisms to dispense the liquid, which is not a precise mechanism for dispensing medicine. Additionally, such devices may be large and not compact, which makes it more difficult for patients to use.

Therefore, there is a need for a liquid dispenser device that is suitable for use with therapeutic liquids that precisely and accurately dispenses a volume of liquid to a patient. There is also a need for such a device that can prevent administration of the therapeutic liquid more frequently than recommended or prescribed. There is also a need for a liquid dispenser device that records administration of dosages of a therapeutic liquid, thereby enabling a user or the user's medical professional to track usage and optimize treatment. There is a further need for such a device to be portable.

### BRIEF SUMMARY OF INVENTION

It is therefore an aspect of the present invention to provide a reusable liquid dispensing device. The device is adapted to receive cartridges containing a liquid substance and may include a plurality of doses of the liquid substance. The device may be adapted to allow the use of a plurality of cartridges with different types of substances in a predetermined time frame. It is a further aspect of the device to regulate the specific amount of a substance (dosage) dispensed from the device by a patient and to regulate the time intervals of those dosages. The device is adapted to accept parameters for dosage volume and interval from a physician or other authorized medical personnel. A doctor would recommend a maximum dosage and minimum time interval between doses for that patient. Based on medical recommendations for that individual, the device will not allow the patient to dispense more than programmed and it will not allow the patient to use the device within a certain time frame. A locking cap mechanism may be securely placed over the end of the cartridge, preventing medicine from being extracted by the patient.

In general, the present invention provides a device comprising a housing which surrounds and secures the mechanism of liquid delivery of the device. The housing comprises at least one exit opening, at least one control button and a display screen. The mechanism for delivering a liquid includes a translational screw that is in contact with and capable of being driven by a micro-motor and a cavity adapted to engage a liquid-containing cartridge. The at least one exit opening is in fluid communication with the liquid-containing cartridge when the liquid-containing cartridge is engaged in the cavity.

In one example, the mechanism of the dispenser device additionally comprises control circuitry. The control circuitry may include a chip reader adapted to receive information from a chip provided as a part of the liquid-containing cartridge. The control circuitry may control the micro-motor, and may establish a maximum dosage and a minimum time interval between doses. In some examples, the maximum dosage and minimum time interval between doses are outside of the control of the user of the device, that is, only authorized medical personnel may control the maximum dosage and minimum time interval between doses. In some examples, the user/patient may set a dosage less than the maximum dosage or at a greater time interval from the previous dose than the minimum time interval between doses. The cartridge may include a locking cap mechanism to prevent extraction of the liquid from the liquid-filled cartridge.

The device may be used in a method of administration of a liquid to an individual. The method includes providing a dispenser device to the individual, wherein the dispenser device comprises a housing and a mechanism for delivering a liquid. The mechanism is located within the housing, which comprises at least one exit opening, at least one control button and a display screen. The mechanism for delivering a liquid comprises a translational screw in contact with a micro motor, a cavity adapted to engage a liquid-containing cartridge, and the at least one exit opening. The dispenser device is adapted to receive control instructions. The method additionally includes providing a liquid-filled cartridge adapted to be inserted into the cavity such that the cartridge can be placed in contact with the translational screw, inserting the liquid-filled cartridge into the dispenser device, and engaging the dispenser device to provide a liquid to the individual.

According to such a method, the dispenser device may include a chip reader and the liquid-filled cartridge may include a memory chip adapted to carry control instructions which can be delivered to the dispenser device from the memory chip. In some examples, the control instructions are provided by an authorized technician other than the user/patient consuming the liquid contained in the device. In some examples, the maximum dosage and minimum time interval between doses are outside of the control of the user of the device, that is, only authorized medical personnel may control the maximum dosage and minimum time interval between doses. In some examples, the user/patient may set a dosage less than the maximum dosage or at a greater time interval from the previous dose than the minimum time interval between doses. The cartridge may also include a locking cap mechanism to prevent extraction of the liquid from the liquid-filled cartridge. In some examples, the micro-motor is activated to dispense a predetermined amount of liquid from the liquid-filled cartridge in response to the control instructions such as the dosage and time interval set by the user. In addition or in the alternative, the liquid-containing cartridge may include a locking cap mechanism.

In one particular example, the patient turns on the device, and then inserts the cartridge. The cartridge has a memory chip, and the device has a memory chip reader. When the cartridge is inserted into the device, the memory chip relays information to the reader, including the type of (liquid) substance within the cartridge, opening the locking cap on the cartridge, and patient information. Patient information could include a patient ID/tag, the patient's doctor recommended dosages, and a doctor recommended time interval for those dosages.

The patent chooses a specific dosage (below or equal to the maximum doctor recommended amount) using the interface. Then the patient can choose a specific time interval to be notified about the next dosage (within doctor recommendation). The device will not allow the patient to choose any settings that are not within doctor recommended parameters. The patient finalizes their settings and can choose to save those settings for future use (“One Click Use”).

When settings are finalized for that dispensing session, the patient presses the dispensing button and a micro motor turns a translational screw, which pushes forward a syringe plunger through the cartridge. Volume displacement within the cartridge is determined by the number of turns the translational screw makes. Each turn of the screw corresponds to the volume displacement within the cartridge. An encoder is used to monitor the actions of the motor and translational screw, and will stop the motor when the desired number of turns are reached.

As the translational screw pushes the syringe plunger forward, liquid is dispensed from a small nozzle that will lead directly to the mouth piece of the device. The liquid is dispensed through the small nozzle and mouth piece as the translational screw pushes the liquid. Once the motor and translational screw stops, the liquid will no longer be pushed through the small nozzle and mouthpiece. The mouth piece may be replaceable, depending on the patient's preference. Mouthpieces can be tailored specifically for dispensing liquid under tongue, dispensing liquid in food, dispensing liquid in drinks, etc.

The patient is notified once the device finishes dispensing the liquid. After notifying the patient, it can automatically go to sleep or turn off. If the patient sets an alarm using the device, it will alert them for their next dosage. The device will not work if the patient tries to use it before the specific time for their next dosage.

The device may optionally include wireless communication, a micro GPS chip, and/or password protection. Wireless communication may be used sync data to the patient's computer, tablet, or smartphone app. This data could include: the exact dosage, time intervals of those dosages, and time of day of their dispensing sessions, which can be used to track their use of the device. Wireless communication and app communication can be used to set up timers to notify the patient of their next dose. Wireless communication can also be used to communicate with other similar devices, preventing the patient from overusing their devices. The wireless communication between devices prevents overlap in the patient's dosages and time intervals. For security reasons, the micro GPS chip could be used to track a device if it is lost, and a password can be put on the device so that the patient would have to enter a short passcode before using the device.

### BRIEF DESCRIPTION OF THE SEVERAL VIEWS OF THE DRAWINGS

FIG. 1 is a perspective view of a first example of the dispenser of the claimed invention.

FIG. 2 is a cut-away view of the first example of the dispenser of the present invention in a static state, showing a full cartridge containing the liquid to be delivered, installed into the device.

FIG. 3 is a cut-away view of the first example of the dispenser of the present invention during use, showing a partially used cartridge containing the liquid to be delivered, installed into the device.

FIG. 4 is a cut-away view of a second example of the dispenser of the present invention, in which the micromotor, translational screw and liquid-filled cartridge are not in-line axially, but are offset.

### DETAILED DESCRIPTION OF THE INVENTION

As stated above, the present invention is directed to a reusable liquid dispenser that is adapted to deliver a predetermined amount of a liquid substance to an individual. The invention may be understood more fully with reference to the following illustrative examples. These examples, however, should not be viewed as limiting the scope of the invention, but as a representative basis for understanding various aspects of the invention. The claims will serve to define the invention.

It is to be understood therefore, that reusable liquid dispenser may vary in size and/or shaped depending upon the particular application it is designed for, or even merely for aesthetic purposes. Likewise, differently configured arrangements of parts may fall within the scope of the present invention. For example, the examples shown in the drawings are not necessarily to scale and may vary in the configuration and/or placement of one or more components.

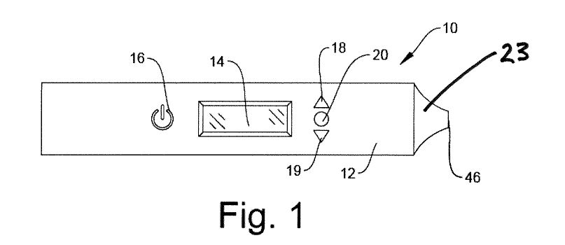

Referring to the drawings, wherein like reference numerals refer to like elements, one example of the present invention is shown in FIGS. 1-3. As shown in FIG. 1, the liquid dispenser device 10 comprises a housing 12 which surrounds and secures the mechanism of liquid substance delivery of the device. Housing 12 includes an exit aperture 46 in mouthpiece 23. The housing may include a power button 16 to switch the device on or off, a display screen 14 and one or more control buttons (18, 19 and 20, respectively) for entering information or changing the display provided on display screen 14.

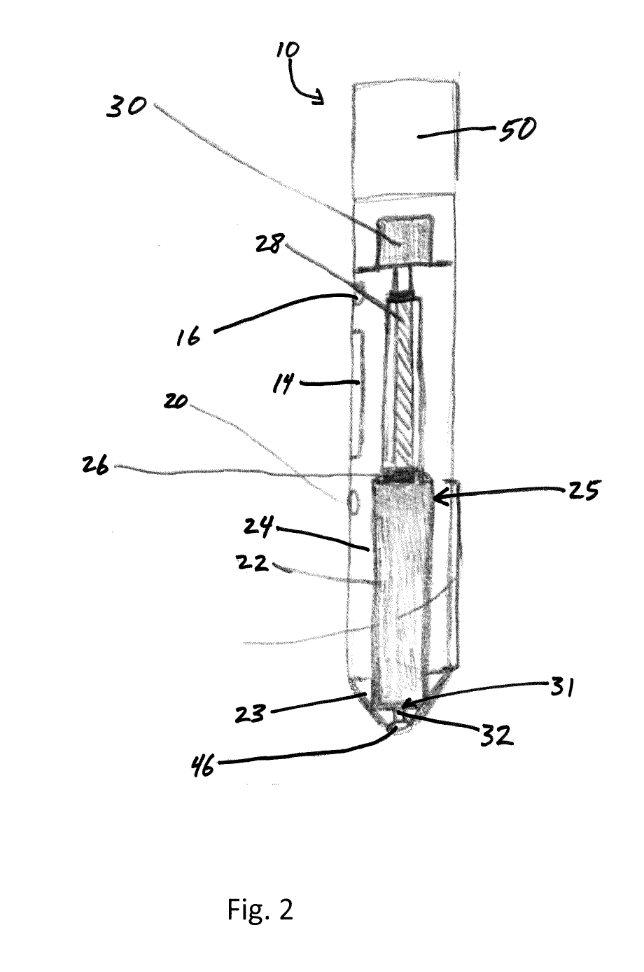

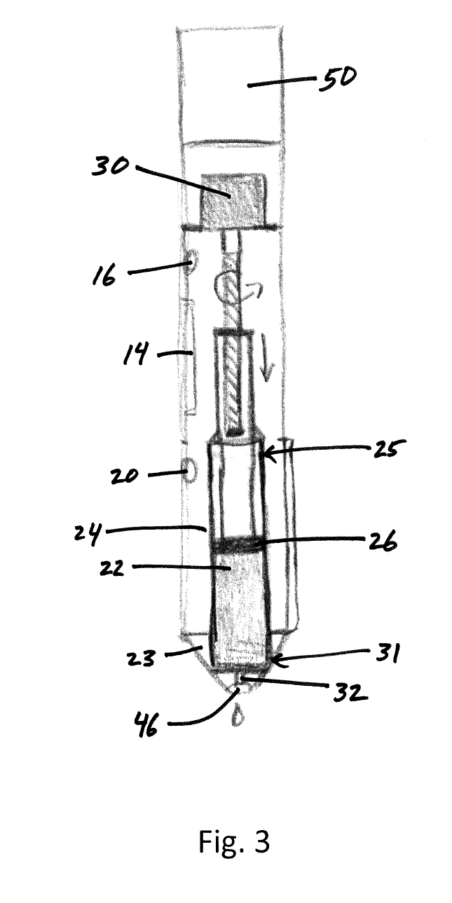

FIGS. 2 and 3 show a partial cut-away view of the liquid dispenser shown in FIG. 1. A cartridge 22 containing the liquid to be dispensed to the individual is installed in a cavity 24 adapted to receive cartridge 22. Cartridge 22 may be reusable or disposable. Cartridge 22 has a first end 25 which includes a syringe type plunger 26. Plunger 26 can be extended into the interior of cartridge 22 by translational screw 28 which first end 25 contacts when engaged. Translational screw 28 in turn is controlled by micro-motor 30, which is activated and deactivated by internal control circuitry (not shown) to deliver a predetermined volume of the contents of cartridge 22 to exit aperture 46 of mouthpiece 23.

Cartridge 22 has a second end 31 opposite to first end 25 of cartridge 22. Second end 31 has an aperture 32 covered with a locking cap mechanism (not shown). Aperture 32 is in fluid communication with exit aperture 46 of mouthpiece 23.

The liquid dispenser further utilizes a battery 50 to power the internal circuitry (not shown), display 14, and any signals provided to the user, whether auditory, visual or tactile (such as by vibration). Battery 50 may be a rechargeable battery as known in the art.

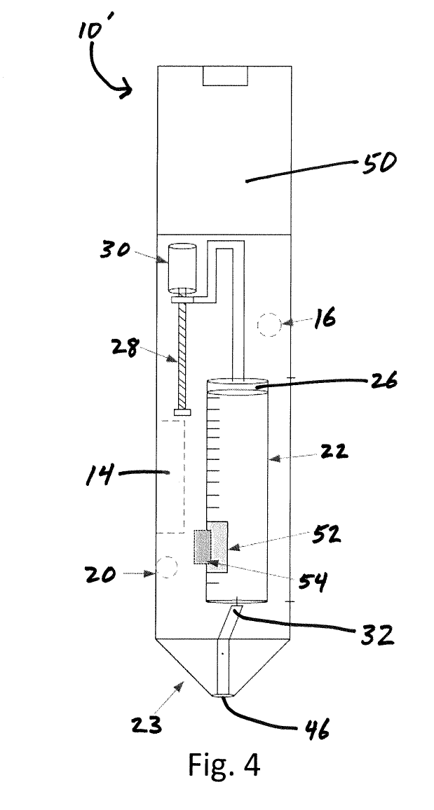

An additional example of the volume displacement dosage dispenser 10′ of the present invention is provided in FIG. 4. The example provided in FIG. 4 is similar to the example provided in FIGS. 1-3 except that the translational screw 28 and micro-motor 30 are offset from cartridge 22. As with the previous example, dispenser 10′ comprises a housing 12, an exit aperture 46 in mouthpiece 23, a power button 16 to switch the device on or off, a display screen 14 and one or more control buttons (20) for entering information or changing the display provided on display screen 14. Additionally, cartridge 22 has a first end 25 which includes a syringe type plunger 26. Plunger 26 can be extended into the interior of cartridge 22 by translational screw 28 which is controlled by micro-motor 30, which is activated and deactivated by internal control circuitry (not shown) to deliver a predetermined volume of the contents of cartridge 22 to mouthpiece 23. A memory chip 52, provided as a component of cartridge 22, is shown in the example of FIG. 4. Memory chip 52 may be programmed by a doctor or other authorized medical staff with dosing parameters such as maximal and/or minimal dose and timing of doses. Device 10′ includes a memory chip reader 54, adapted to read memory chip 52, when cartridge 22 is inserted into device 10′. Memory chip 52 can contain information such as the substance within the cartridge, the concentration of the liquid, and patient information. The patient information can include a specific ID/tag for that patient, a doctor recommended dose of medication, and a doctor recommended time frame for those dosages. The memory chip is programmed to notify the memory chip reader to unlock the locking cap mechanism.

Operation of liquid delivery device 10 or 10′ may also be described with reference to FIGS. 1-4. The user may turn device 10/10′ on by pressing power button 16 down for a predetermined period of time, such as 3-5 seconds. The user will have been previously provided with cartridge 22 adapted for use with device 10 or 10′. Cartridge 22 may be reusable or disposable, and may be designed to contain a plurality of doses of liquid.

First end 25 of cartridge 22 contains a syringe type plunger 26, while second end 31 has a thin nozzle or aperture 32 covered with a locking cap mechanism (not shown) or other similar mechanism to prevent the contents of cartridge 22 to be extracted outside of the control of the liquid dispenser device 10. The cartridge may include a memory chip 52, which interacts with a memory chip reader 54 within the device 10/10′ specifically for that patient. Memory chip 52 on cartridge 22 can contain information about the substance within the cartridge, the concentration of the liquid, and patient information. The patient information can include a specific ID/tag for that patient, a doctor recommended dose of medication, and a doctor recommended time frame for those dosages. The memory chip is programmed to notify the memory chip reader to unlock the locking cap mechanism.

The patient inserts cartridge 22 into device 10 (if first time use for that cartridge). The memory chip reader receives information from the memory chip on the cartridge. The memory chip informs the reader of the type of liquid within the cartridge, the concentration of the liquid, and patient information. Patient information received by the device includes a patient ID/tag, doctor recommended dosages, and doctor recommended time frame for those dosages. The device may be programmed to lock out the user if the specific patient ID/tag does not match the device (e.g. if the patient tries to use another patient's cartridge, another company's cartridge, a homemade cartridge).

Options on the interface/display screen 14 may appear, notifying the patient of what type and concentration of liquid in the cartridge. Depending on the contents, warnings about the substance may optionally appear. The patient can then choose a specific dosage using control buttons 18, 19, 20, but will not be able to go beyond the maximum recommended by their doctor. The patient can then choose a specific time frame for their next dosage as long as it is within their doctor's recommended time frame. The patient may have the option to set a timer to notify of the availability of the next dosage. The patient finalizes their settings for that session and may have the option to save settings for future use (“One Click Use”).

The patient presses a dispensing button and micro-motor 30 turns on, which turns a translational screw 28 or similar structure. Translational screw 28 engages cartridge 22 via plunger 26. It is envisioned that other configurations and structures of this mechanism may accomplish the same dosage. Micro-motor 30 includes an encoder which monitors the rotations of translational screw 28 to determine the distance traveled by translational screw 28. The distance traveled is directly correlated to the volume displacement within cartridge 22. Variables of volume displacement include the diameter of the cartridge and the pitch of the screw. The encoder will stop micro-motor 30 and translational screw 28 when the displacement of liquid within cartridge 22 equals the specific dosage.

As the force of translational screw 28 pushes plunger 26 forward, the liquid within the cartridge is pushed through aperture 32 of second end 31 of cartridge 22. The liquid cannot escape/drip from the cartridge without the force of translational screw 28 and syringe plunger 26. The liquid leaving aperture 32 leads directly to exit aperture 46 of mouthpiece 23, allowing the liquid to flow out of the device. Mouthpiece 23 may be replaceable, and it is envisioned that many types of options for mouthpiece 23 depending on the patient's specific needs. For example, it may support mouthpieces specifically for dispensing liquid under the tongue, in food, in drinks, etc.

When the volume displacement mechanism is finished pushing the liquid through the small nozzle/mouthpiece of the device, the patient is notified that the device is finished. The patient may notified by a message or signal on display 14. Alternatively, the notification may be an auditory signal such as one or more tones sounded, or a vibrational signal.

The present invention provides a precisely measured dosage so the patient only has to consume the liquid that has exited the device to obtain a well-defined dosage. After the patient has finished consuming the liquid dispensed from the device, the power button may be pressed to put it to sleep/into a stand-by mode. If the patient does not put it to sleep, it may automatically go to sleep after a certain amount of time. If the patient set a timer for their next dosage, the device may be programmed to wake up and an alarm will alert the patient to the availability of another dosage. When the patient is finished with the device, the device can be turned off fully by holding the power button for a predetermined period of time, such as 3-5 seconds.

Optionally, display 14 may provide additional information such as remaining battery life and the amount of liquid left in the cartridge. The device (and other devices) may be programmed such that it cannot be used outside of the patient's doctor recommended time frame for the next dosage. This ensures the patient will not exceed their maximum doctor recommended dosage, which helps to prevent overdosing.

This device may also have wireless communication capabilities. Wireless communication can be used for this device to communicate with other corresponding devices. This ensures that the patient is not using more than their doctor recommended dosage and time frame. The wireless communication capabilities of this device can also be used to sync data with smartphone apps, tablet apps, or computer apps. Wireless communication can be used to manage the device from the patient's smartphone, tablet, or computer, allowing them to turn on/off the device and choose settings for the device. If the patient will use the same settings for that specific cartridge, the patient can save their settings in the app for “One Click Use” later on. Also, using wireless communication, the patient's smartphone, tablet, or computer app can notify them when they are allowed to use the device and can notify the patient for their next dosage (rather than using the device alarm).

Using a charger cord such as a USB cable and corresponding port for the battery, the patient can connect the liquid dispenser device to their computer manually. For security, the device also has the option to be password protected. In this case, the patient would have to type in a short password to use the device. It is also envisioned that the device can have a micro GPS chip. If the patient loses their device or if it is stolen, the device could be tracked using the existing GPS systems.

Based upon the foregoing disclosure, it should now be apparent that the volume displacement dosage liquid dispenser will carry out the objects set forth hereinabove, and may do so in a manner that allows the device to hand-held or otherwise portable. It is, therefore, to be understood that any variations evident fall within the scope of the claimed invention and thus, the selection of specific component elements can be determined without departing from the spirit of the invention herein disclosed and described.

I claim:

1. A dispenser device for the administration of a substance to a user as a liquid, the dispenser device comprising:
a housing;
a mechanism for delivering a liquid, wherein the mechanism is located within the housing;
wherein the housing comprises at least one exit opening, at least one control button and a display screen;
wherein the mechanism for delivering a liquid comprises a translational screw in contact with a micro motor, a cavity adapted to engage a liquid-containing cartridge, and wherein the at least one exit opening is in fluid communication with the liquid-containing cartridge when the liquid-containing cartridge is engaged in the cavity.

2. The dispenser device of claim 1, wherein the mechanism for delivering a liquid additionally comprises control circuitry.

3. The dispenser device of claim 2, wherein the control circuitry comprises a chip reader adapted to receive information from a chip provided with the liquid-containing cartridge.

4. The dispenser device of claim 3, wherein the micro motor is controlled by control circuitry according to information provided by the chip reader.

5. The dispenser device of claim 4, wherein the control circuitry establishes a maximum dosage and a minimum time interval between doses.

6. The dispenser device of claim 5, wherein the maximum dosage and time intervals between doses are outside of the control of the user of the device.

7. The dispenser device of claim 6, wherein the user may set a dosage less than the maximum dosage or at a greater time interval from the previous dose than the minimum time interval between doses.

8. The dispenser device of claim 4, wherein the liquid-containing cartridge additionally comprises a locking cap mechanism.

9. A method of administration of a liquid to an individual, the method comprising:
providing a dispenser device to the individual, wherein the dispenser device comprises a housing and a mechanism for delivering a liquid, wherein the mechanism is located within the housing;
wherein the housing comprises at least one exit opening, at least one control button and a display screen;
wherein the mechanism for delivering a liquid comprises a translational screw in contact with a micro motor, a cavity adapted to engage a liquid-containing cartridge, and the at least one exit opening, and wherein the dispenser device is adapted to receive control instructions;
providing a liquid-filled cartridge adapted to be inserted into the cavity such that the cartridge can be placed in contact with the translational screw;
inserting the liquid-filled cartridge into the dispenser device; and
engaging the dispenser device to provide a liquid to the individual.

10. The method of claim 9, wherein the dispenser device comprises a chip reader and wherein the liquid-filled cartridge additionally comprises a memory chip adapted to carry control instructions and further wherein the control instructions are provided to the dispenser device from the memory chip.

11. The method of claim 10, wherein the control instructions are provided by an authorized technician other than the user.

12. The method of claim 11, wherein the control instructions provide a maximum dosage and a minimum time interval between doses.

13. The method of claim 12, wherein the user may set a dosage less than the maximum dosage or greater than the minimum time interval between doses.

14. The method of claim 13, wherein the micro motor is activated to dispense a predetermined amount of liquid from the liquid-filled cartridge in response to the dosage and time interval set by the user.

15. The method of claim 14, wherein the micro motor is controlled by the control instructions.

16. The method of claim 112, wherein the micro motor is activated to dispense a predetermined amount of liquid from the liquid-filled cartridge in response to the dosage and time interval set by the user.

17. The method of claim 12, wherein the micro motor are controlled by the control instructions.

18. The method of claim 17, wherein the dispenser device is additionally adapted to wirelessly communicate with other similar devices, a computer, a smartphone or a tablet.

19. The method of claim 9, wherein the dispenser device is additionally adapted to wirelessly communicate with other similar devices, a computer, a smartphone or a tablet.

20. The method of claim 9, wherein the liquid-containing cartridge additionally comprises a locking cap mechanism.
